(19) United States
(12) Patent Application Publication
(10) Pub. No.: US 2020/0405671 A1
(43) Pub. Date: Dec. 31, 2020

(54) METHODS AND MATERIALS FOR TREATING POLYCYSTIC KIDNEY DISEASE

(71) Applicant: Mayo Foundation for Medical Education and Research, Rochester, MN (US)

(72) Inventors: Maria V. Irazabal Mira, Rochester, MN (US); Vicente E. Torres, Rochester, MN (US)

(73) Assignee: Mayo Foundation for Medical Education and Research, Rochester, MN (US)

(21) Appl. No.: 16/911,932

(22) Filed: Jun. 25, 2020

Related U.S. Application Data
(60) Provisional application No. 62/866,210, filed on Jun. 25, 2019.

(51) Int. Cl. A61K 31/195; A61K 31/55; A61K 31/551; A61P 13/12

(52) U.S. Cl. CPC A61K 31/195; A61P 13/12; A61K 31/551; A61K 31/55

(57) ABSTRACT

This document relates to methods and materials for treating polycystic kidney disease (PKD) and/or a complication associated with a PKD (e.g., a cardiovascular complication). For example, one or more betaines (e.g., trimethylglycine) can be administered to a mammal having, or at risk of developing, a PKD to treat the mammal.

### CROSS-REFERENCE TO RELATED APPLICATIONS

This application claims the benefit of U.S. Patent Application Ser. No. 62/866,210, filed on Jun. 25, 2019. The disclosure of the prior application is considered part of (and is incorporated by reference in) the disclosure of this application.

### BACKGROUND

### 1. Technical Field

This document relates to methods and materials for treating a polycystic kidney disease (PKD) and/or a complication associated with a PKD (e.g., a cardiovascular complication). For example, one or more betaines (e.g., trimethylglycine) can be administered to a mammal having, or at risk of developing, a PKD to treat the mammal.

### 2. Background Information

Autosomal dominant PKD (ADPKD) is a systemic disorder where progressive development and enlargement of kidney cysts can lead to end-stage renal disease (ESRD), and can be associated with multiple extra renal manifestations, including manifestations in the heart and vasculature. Cardiovascular mortality remains the leading cause of death in ADPKD (Helal et al., Am. J. Nephrology, 36(4):362-70 (2012)). Over 50% of patients with ADPKD eventually develop end stage kidney disease and require dialysis or kidney transplantation (Tones et al., Lancet, 369(9569):1287-1301 (2007); and Grantham et al., N. Engl. J. Med., 359(14):1477-1485 (2008)).

### SUMMARY

This document provides methods and materials for treating a mammal (e.g., a human) having, or at risk of developing, a PKD such as ADPKD. For example, this document provides methods and materials for using one or more betaines (e.g., trimethylglycine) to treat a mammal having, or at risk of developing, a PKD such as ADPKD. In some cases, a mammal having, or at risk of developing, a PKD can be administered a composition including one or more betaines to treat the mammal.

As demonstrated herein, betaines can be used to treat a PKD such as ADPKD. For example, 1% and 2% betaine supplementation can increase plasma and tissue betaine concentrations, can reduce kidney/body weight, and/or can reduce cystic index in PKD mice. Having the ability to reduce or eliminate one or more symptoms of a PKD and/or one or more complications associated with a PKD can provide a unique and unrealized opportunity to treat a PKD such as ADPKD. For example, a mammal having, or at risk of developing, a PKD such as ADPKD can be treated by administering a betaine.

In general, one aspect of this document features a method for treating a mammal having a polycystic kidney disease (PKD). The method comprises (or consists essentially of or consists of) administering a composition comprising one or more betaines to the mammal to reduce a cystic index in the mammal. The method can comprise identifying the mammal as being in need of a treatment for the PKD. The mammal can be a human. The PKD can be an ADPKD. The composition can comprise trimethylglycine. The composition can comprise from about 1 gram to about 20 g of the trimethylglycine. The composition can comprise one or more vasopressin receptor antagonists. The composition can comprise tolvaptan or lixivaptan.

In another aspect, this document features a method for treating a mammal having a PKD. The method comprises (or consists essentially of or consists of) administering a composition comprising one or more betaines to the mammal to reduce a symptom of the PKD in the mammal. The method can comprise identifying the mammal as being in need of a treatment for the PKD. The mammal can be a human. The PKD can be an ADPKD. The symptom can be selected from the group consisting of back pain, side pain, headache, a feeling of fullness, an enlarged kidney, blood in the urine, high blood pressure, kidney failure, mitral valve prolapse, diverticulosis, brain aneurysm, and endothelial dysfunction (ED). The composition can comprise trimethylglycine. The composition can comprise from about 1 g to about 20 g of the trimethylglycine. The composition can comprise one or more vasopressin receptor antagonists. The composition can comprise tolvaptan or lixivaptan.

In another aspect, this document features a method for treating a mammal having a PKD. The method comprises (or consists essentially of or consists of) administering a composition comprising one or more betaines to the mammal to increase a concentration of renal betaine in the mammal. The method can comprise identifying the mammal as being in need of a treatment for the PKD. The mammal can be a human. The PKD can be an ADPKD. The composition can comprise trimethylglycine. The composition can comprise from about 1 g to about 20 g of the trimethylglycine. The composition can comprise one or more vasopressin receptor antagonists. The composition can comprise tolvaptan or lixivaptan.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1: (A) Homocysteine metabolic pathway. 5-MTHF: 5-methyltetra hydrofolate; THF: tetra hydrofolate; MS: methionine synthase; SAM: S-adenosylmethionine; SAH: S-adenosylhomocysteine; DMG: Dimethylglycine; DD: dimethylglycine dehydrogenase; SD: sarcosine dehydrogenase; SHMT: serine hydroxymethyltransferase; BAD: Betaine aldehyde dehydrogenase; CO: Choline oxidase; BHMT: betaine-homocysteine-methyltransferase; CBS: cystathione β-synthase. (B) Schematic concept depicting the role of impaired betaine dependent re-methylation and elevated levels of Hcy in endothelial dysfunction in ADPKD. Increased urine excretion of betaine and/or disruption of choline oxidation to betaine lead to betaine insufficiency, impaired betaine re-methylation and elevation of Hcy. Hcy elevation contribute to increased NOX4, increased ROS production and mitochondrial dysfunction further increasing ROS production. Increased cellular ROS leads to decreased NO availability, in addition to disruption of cell proliferation and apoptosis, inducing ED and contributing to renal disease progression.

FIG. 2: ¹HNMR spectroscopy depicting stability of betaine in water during the course of 1 week. Three spectral lines on top indicating 2% betaine represent betaine concentration as determined by ¹HNMR spectroscopy at day 0, day, 2 and day 8. Below, three spectral lines indicating 1% betaine represent betaine concentration as determined by ¹HNMR spectroscopy at day 0, day, 2 and day 8. At the bottom, flat spectra can be seen for the animals receiving control water without betaine, indicated as control day 2 and 8. Overall, betaine was stable and non-significant changes in betaine concentration were observed during the course of one week.

FIG. 3: ¹HNMR spectroscopy depicting tissue betaine concentration in the different animal groups. Spectra for animals receiving 2% betaine are colored in green, 1% betaine in blue, and control animals with no betaine supplementation in red. Supplementation of betaine at both, 1 and 2%, resulted in significant increases of tissue betaine concentration.

FIG. 4: Histological images from representative animals from each treatment group. Representative hematoxylin and eosin-stained kidney mid-sections from Pkd1^{RC/RC} mice on 1% betaine, or 2% betaine or no betaine in drinking water. Betaine supplementation reduced cystic index to 11.1% (1% betaine) and 9.4% (2% betaine) vs 21.1% (no betaine). Cystic index is calculated as the percentage of cyst area (white rounded areas) within the total kidney section.

FIG. 5: Correlation between tissue betaine concentration and kidney weight/body weight (KW/BW). In ADPKD, the higher the KW/BW index, the more severe the disease is. There was an inverse correlation between the concentration of betaine achieved in the kidney with betaine supplementation, and the KW/BW of the animals at the time of euthanasia suggesting the higher betaine concentrations are associated with amelioration of the disease.

FIG. 6: Intrarenal microvasculature in ADPKD with and without betaine treatment (control) and in wild type (WT) animals at 6 months of age in male animals (A) and female animals (B). Capillaries were identified by the presence of lumen, red blood cells, and/or an endothelial cell lining, and the ratio of capillary number to non-cystic parenchyma was calculated.

FIG. 7: Mean Arterial Pressure (MAP) in ADPKD with and without betaine treatment (control) and in wild type (WT) animals at 1 and 6 months of age in male animals (A) and female animals (B). Comparisons for all pairs were done using Tukey-Kramer. For all groups n=8 per sex except for WT n=4. *p<0.05 vs CTRL. †p=n.s. vs WT.

### DETAILED DESCRIPTION

This document provides methods and materials for treating a mammal (e.g., a human) having, or at risk of developing, a PKD such as ADPKD. For example, this document provides methods and materials for using one or more betaines (e.g., trimethylglycine) to treat a mammal having, or at risk of developing, a PKD such as ADPKD. In some cases, a mammal having, or at risk of developing, a PKD can be administered a composition including one or more betaines to treat the mammal.

In some cases, a mammal (e.g., a human) having, or at risk of developing, a PKD such as ADPKD can be administered one or more betaines (e.g., trimethylglycine) to reduce or eliminate one or more symptoms of a PDK and/or one or more complications associated with a PKD. Examples of symptoms of a PDK and complications associated with PKD include, without limitation, back pain, side pain, headache, a feeling of fullness (e.g., in the abdomen), increased size of the abdomen (e.g., due to an enlarged kidney), blood in the urine, high blood pressure, loss of kidney function (e.g., kidney failure), heart valve abnormalities (e.g., mitral valve prolapse), colon problems (e.g., diverticulosis), development of an aneurysm (e.g., a brain aneurysm), and ED. For example, one or more betaines can be administered to a mammal (e.g., a human) in need thereof (e.g., a human having, or at risk of developing, a PKD) as described herein to reduce the severity of one or more symptoms of a PDK and/or one or more complications associated with PKD by, for example, 10, 20, 30, 40, 50, 60, 70, 80, 90, 95, or more percent.

In some cases, a mammal (e.g., a human) having, or at risk of developing, a PKD such as ADPKD can be administered one or more betaines (e.g., trimethylglycine) to reduce or eliminate one or more cysts (e.g., one or more renal cysts) within the mammal. For example, one or more betaines can be administered to a mammal (e.g., a human) in need thereof (e.g., a human having one or more cysts associate with a PKD) as described herein to reduce the size (e.g., volume) of a cyst within the mammal by, for example, 10, 20, 30, 40, 50, 60, 70, 80, 90, 95, or more percent. For example, one or more betaines can be administered to a mammal (e.g., a human) in need thereof (e.g., a human having one or more cysts associated with PKD) as described herein to reduce the cystic index (also referred to as a cystic burden; e.g., the percentage of an organ such as a kidney that is occupied by cysts) in the mammal by, for example, 10, 20, 30, 40, 50, 60, 70, 80, 90, 95, or more percent. Any appropriate method can be used to determine the size of a cyst (e.g., a renal cyst) and/or a cystic index within a mammal (e.g., a mammal having, or at risk of developing, a PKD). For example, ultrasound, computed tomography (CT) scanning, magnetic resonance imaging (MRI), and/or histological images can be used to determine the size of a cyst (e.g., a renal cyst) and/or a cystic index of a mammal (e.g., a mammal having, or at risk of developing, a PKD). In some cases, a cystic index can be determined as described elsewhere (see, e.g., Irazabal et al., J. Vis. Exp., (100):e52757 (2015); and Hopp et al., J. Clin. Invest., 122(11):4257-42-73 (2012)).

In some cases, a mammal (e.g., a human) having, or at risk of developing, a PKD such as ADPKD can be administered one or more betaines (e.g., trimethylglycine) to reduce the weight of one or both kidneys within the mammal and/or to reduce the body weight of the mammal. For example, one or more betaines can be administered to a mammal (e.g., a human) in need thereof (e.g., a human having one or more cysts associate with a PKD) as described herein to reduce the weight of a kidney within the mammal and/or to reduce the body weight of the mammal by, for example, 10, 20, 30, 40, 50, 60, 70, 80, 90, 95, or more percent. Any appropriate method can be used to determine the weight of a kidney.

In some cases, a mammal (e.g., a human) having, or at risk of developing, a PKD such as ADPKD can be administered one or more betaines (e.g., trimethylglycine) to reduce the volume of one or both kidneys within the mammal. For example, one or more betaines can be administered to a mammal (e.g., a human) in need thereof (e.g., a human having one or more cysts associate with a PKD) as described herein to reduce the volume of a kidney within the mammal by, for example, 10, 20, 30, 40, 50, 60, 70, 80, 90, 95, or more percent. Any appropriate method can be used to determine the volume of a kidney. For example, ultrasound, computed tomography (CT) scanning, magnetic resonance imaging (MM) can be used to determine the volume of a kidney.

In some cases, a mammal (e.g., a human) having, or at risk of developing, a PKD such as ADPKD can be administered one or more betaines (e.g., trimethylglycine) to preserve (e.g., maintain) the vasculature (e.g., a capillary count) within the mammal. For example, one or more betaines can be administered to a mammal (e.g., a human) in need thereof (e.g., a human having one or more cysts associate with a PKD) as described herein to preserve the vasculature within the mammal. In some cases, a mammal (e.g., a human) having, or at risk of developing, a PKD such as ADPKD can be administered one or more betaines (e.g., trimethylglycine) to increase the vasculature (e.g., a capillary count) within the mammal. For example, one or more betaines can be administered to a mammal (e.g., a human) in need thereof (e.g., a human having one or more cysts associate with a PKD) as described herein to increase the vasculature (e.g., a capillary count) within mammal by, for example, 10, 20, 30, 40, 50, 60, 70, 80, 90, 95, or more percent. Any appropriate method can be used to detect the vasculature (e.g., the capillaries) within a mammal.

In some cases, a mammal (e.g., a human) having, or at risk of developing, a PKD such as ADPKD can be administered one or more betaines (e.g., trimethylglycine) to increase a concentration of betaine in the mammal. An increase in concentration of betaine can be in any appropriate tissue and/or organ of the mammal. Examples of tissues and/or organs in which a concentration of betaine can be increased as described herein (e.g., by administering one or more betaines such as trimethylglycine) include, without limitation, blood (e.g., plasma), kidneys, and liver. In some cases, administering one or more betaines to a mammal having a PKD can be effective to increase the concentration of betaine in one or both kidneys (e.g., can be effective to increase renal betaine) in the mammal. An increased level of betaine can be any level that is higher than a level of betaine that was observed prior to administration of one or more betaine. For example, one or more betaines can be administered to a mammal (e.g., a human) in need thereof (e.g., a human having, or at risk of developing, a PKD) as described herein to increase a concentration of betaine in the mammal by, for example, 10, 20, 30, 40, 50, 60, 70, 80, 90, 95, or more percent.

Any appropriate mammal having, or at risk of developing, a PKD such as ADPKD can be treated as described herein (e.g., by administering one or more betaines such as trimethylglycine). In some cases, a mammal can be a male mammal. In some cases, a mammal can be a female mammal. Examples of mammals having, or at risk of developing, a PKD that can be treated as described herein include, without limitation, humans, non-human primates (e.g., monkeys), dogs, cats, horses, cows, pigs, sheep, mice, and rats. In some cases, a human having, or at risk of developing, a PKD can be treated by administering one or more betaines (e.g., trimethylglycine) to the human.

Any appropriate PKD can be treated as described herein (e.g., by administering one or more betaines such as trimethylglycine). Examples of PKDs that can be treated as described herein include, without limitation, ADPKD and autosomal recessive PKD (ARPKD). In some cases, a mammal (e.g., a human) having, or at risk of developing, ADPKD can be treated by administering one or more betaines (e.g., trimethylglycine) to the mammal.

When treating a mammal having, or at risk of developing, a PKD (e.g., ADPKD) as described herein (e.g., by administering one or more betaines such as trimethylglycine), the mammal can have one or more cysts present in and/or on any tissue or organ within the mammal. Examples of tissues and organs within a mammal having PKD that can have one or more cysts include, without limitation, the kidney, the liver, seminal vesicles, pancreas, and arachnoid membrane. For example, a mammal (e.g., a human) having PKD can have one or more renal cysts (e.g., one or more cysts present on or within one or both kidneys).

In some cases, methods for treating a mammal (e.g., a human) having, or at risk of developing, a PKD such as ADPKD, also can include identifying a mammal as having, or as being at risk of developing, a PKD. Any appropriate method can be used to identify a mammal as having, or as being at risk of developing, a PKD. For example, imaging techniques (e.g., ultrasound, CT scan, and MRI) and/or laboratory tests (e.g., genetic testing for mutation of one or both copies of the polycystic kidney disease gene 1 (PKD1), the polycystic kidney disease gene 2 (PKD2), and/or the polycystic kidney and hepatic disease 1 (PKHD1) gene present in a mammal) can be used to identify a mammal as having, or as being at risk of developing, a PKD such as ADPKD.

Once identified as having, or as being at risk of developing, a PKD such as ADPKD, the mammal (e.g., the human) can be administered, or instructed to self-administer, one or more betaines. Any appropriate betaine can be used as described herein. In general, a betaine is a zwitterion (e.g., a chemical compound having at least one positively charged functional group and at least one negatively charged functional group such that net charge results in a neutral chemical compound) that is involved in methylation reactions and detoxification of homocysteine. Examples of betaines that can be used to treat a mammal having, or at risk of developing, a PKD such as ADPKD as described herein include, without limitation, trimethylglycine, glycine betaine, (carboxymethyl)trimethylammonium inner salt, and oxyneurine. In some cases, a betaine to be used as described herein can be an osmolyte (e.g., an organic osmolyte). In some cases, a betaine to be used as described herein can be a methyl donor. In some cases, a betaine to be used as described herein can be an anhydrous betaine. In some cases, a betaine to be used as described herein can be an aldehyde betaine. In some cases, a betaine to be used as described herein can be in the form of a salt (e.g., a pharmaceutically acceptable salt) such as betaine hydrochloride (betaine-HCl). In some cases, a betaine that can be used as described herein can be trimethylglycine. A chemical formula for a trimethylglycine can be as follows.

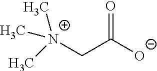

For example, a mammal (e.g., a human) having, or at risk of developing, a PKD such as ADPKD can be treated by administering trimethylglycine to the mammal.

In some cases, one or more betaines (e.g., trimethylglycine) can be formulated into a composition (e.g., a pharmaceutically acceptable composition) for administration to a mammal having, or at risk of developing, a PKD such as ADPKD. For example, one or more betaines can be formulated together with one or more pharmaceutically acceptable carriers (additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, when a composition containing one or more betaines (e.g., trimethylglycine) is administered to a mammal having, or at risk of developing, a PKD such as ADPKD, the composition can be designed for oral or parenteral (including, without limitation, a subcutaneous, intramuscular, intravenous, intradermal, intra-cerebral, intrathecal, or intraperitoneal (i.p.) injection) administration to the mammal. Compositions suitable for oral administration include, without limitation, liquids, tablets, capsules, pills, powders, gels, and granules. In some cases, compositions suitable for oral administration can be in the form of a food supplement. In some cases, compositions suitable for oral administration can be in the form of a drink supplement. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

A composition containing one or more betaines (e.g., trimethylglycine) can include any appropriate amount of the betaine(s). For example, a composition containing one or more betaines can include an amount of betaine(s) that is from about 0.1 percent to about 2.5 percent (e.g., about 1 percent) of the total composition. In some cases, a composition containing trimethylglycine can include 1% trimethylglycine or 2% trimethylglycine.

A composition containing one or more betaines (e.g., trimethylglycine) can be administered to a mammal having, or at risk of developing, a PKD such as ADPKD in any appropriate amount (e.g., any appropriate dose). Effective amounts can vary depending on the route of administration, the age of the subject, the sex of the subject, the general health condition of the subject, excipient usage, the possibility of co-usage with other therapeutic treatments such as use of other agents, and the judgment of the treating physician. An effective amount of a composition containing one or more betaines can be any amount that can treat a mammal having, or at risk of developing, a PKD such as ADPKD as described herein without producing significant toxicity to the mammal. For example, an effective amount of trimethylglycine can be from about 1 gram (g) per day to about 20 g per day. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the PKD in the mammal being treated may require an increase or decrease in the actual effective amount administered.

A composition containing one or more betaines (e.g., trimethylglycine) can be administered to a mammal having, or at risk of developing, a PKD such as ADPKD in any appropriate frequency. The frequency of administration can be any frequency that can treat a mammal having, or at risk of developing, a PKD without producing significant toxicity to the mammal. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition containing one or more betaines (e.g., trimethylglycine) can be administered to a mammal having, or at risk of developing, a PKD such as ADPKD for any appropriate duration. An effective duration for administering or using a composition containing one or more betaines can be any duration that can treat a mammal having, or at risk of developing, a PKD without producing significant toxicity to the mammal. For example, the effective duration can vary from several weeks to several months, from several months to several years, or from several years to a lifetime. In some cases, the effective duration can range in duration from about 10 years to about a lifetime. Multiple factors can influence the actual effective duration used for a particular treatment. For example, an effective duration can vary with the frequency of administration, effective amount, use of multiple treatment agents, and/or route of administration.

In some cases, methods for treating a mammal (e.g., a human) having, or at risk of developing, a PKD such as ADPKD, can include administering to the mammal one or more betaines (e.g., trimethylglycine) as the sole active ingredient to treat the mammal. For example, a composition containing one or more betaines can include the one or more betaines as the sole active ingredient in the composition that is effective to treat a mammal having, or at risk of developing, a PKD.

In some cases, methods for treating a mammal (e.g., a human) having, or at risk of developing, a PKD such as ADPKD as described herein (e.g., by administering one or more betaines such as trimethylglycine) also can include administering to the mammal one or more (e.g., one, two, three, four, five or more) additional active agents (e.g., therapeutic agents) that are effective to treat one or more symptoms of a PKD and/or one or more complications associated with a PKD to treat the mammal. Examples of additional active agents that can be used as described herein to treat one or more symptoms of a PKD and/or one or more complications associated with a PKD include, without limitation, an inhibitor of a vasopressin receptor (e.g., tolvaptan or lixivaptan), angiotensin-converting enzyme (ACE) inhibitors, angiotensin II receptor blockers (ARBs), pain relievers (e.g., acetaminophen), and antibiotics. In some cases, the one or more additional active agents can be administered together with the administration of the one or more betaines (e.g., trimethylglycine). For example, a composition containing one or more betaines also can include one or more additional active agents that are effective to treat one or more symptoms of a PKD and/or one or more complications associated with a PKD. In some cases, a composition can be formulated to include one or more betaines (e.g., trimethylglycine) and one or more vasopressin receptor antagonists (e.g., tolvaptan or lixivaptan) and administered to a mammal (e.g., a human) to treat a PKD such as ADPKD. In some cases, the one or more additional active agents that are effective to treat one or more symptoms of a PKD and/or one or more complications associated with a PKD can be administered independent of the administration of the one or more betaines (e.g., trimethylglycine). When the one or more additional active agents that are effective to treat one or more symptoms of a PKD and/or one or more complications associated with a PKD are administered independent of the administration of the one or more betaines, the one or more betaines can be administered first, and the one or more additional active agents that are effective to treat one or more symptoms of a PKD and/or one or more complications associated with a PKD performed second, or vice versa.

In some cases, methods for treating a mammal (e.g., a human) having, or at risk of developing, a PKD such as ADPKD as described herein (e.g., by administering one or more betaines such as trimethylglycine) also can include subjecting the mammal one or more (e.g., one, two, three, four, five or more) additional treatments (e.g., therapeutic interventions) that are effective to treat one or more symptoms of a PKD and/or one or more complications associated with a PKD to treat the mammal. Examples of additional treatments that can be used as described herein to treat one or more symptoms of a PKD and/or one or more complications associated with a PKD include, without limitation, consuming a restricted diet (e.g., a diet low in methionine, high in choline, and/or high in betaine content), maintaining a healthy body weight, exercising regularly, undergoing dialysis, and undergoing a kidney transplant. In some cases, the one or more additional treatments that are effective to treat one or more symptoms of a PKD and/or one or more complications associated with a PKD can be performed at the same time as the administration of the one or more betaines (e.g., trimethylglycine). In some cases, the one or more additional treatments that are effective to treat one or more symptoms of a PKD and/or one or more complications associated with a PKD can be performed before and/or after the administration of the one or more betaines (e.g., trimethylglycine).

In some cases, methods for treating a mammal (e.g., a human) having, or at risk of developing, a PKD such as ADPKD as described herein (e.g., by administering one or more betaines such as trimethylglycine) also can include monitoring the mammal being treated. For example, methods described herein also can include monitoring the severity or progression of a PKD such as ADPKD in a mammal. Any appropriate method can be used to monitor the severity or progression of a PKD in a mammal. In some cases, one or more symptoms of PKD and/or one or more complications associate with a PKD can be assessed using any appropriate methods and/or techniques, and can be assessed at different time points. For example, imaging or spectroscopic techniques (e.g., ultrasound, CT scan, MRI, or 1HNMR spectroscopy) can be used to assess the presence, absence, or size of a cyst (e.g., renal cyst). In some cases, any appropriate urine and plasma biomarker can be used to monitor the severity or progression of a PKD in a mammal. For example, one or more symptoms of PKD and/or one or more complications associate with a PKD can be assessed using any appropriate urine and plasma biomarker and can be assessed at different time points. In some cases, one or more urine and/or plasma biomarkers using any common technique (e.g., 1HNMR, Mass spectroscopy, or ELISA) can be used to assess the presence, absence, or size of a cyst (e.g., renal cyst).

In some cases, methods for treating a mammal (e.g., a human) having, or at risk of developing, a PKD such as ADPKD as described herein (e.g., by administering one or more betaines such as trimethylglycine) also can include monitoring the treatment response. Any appropriate urine and/or plasma biomarker can be used to monitor the treatment response of a PKD in a mammal. In some cases, one or more metabolic or cellular pathways can be assessed using any appropriate urine and/or plasma biomarker and can be assessed at different time points. For example, urine and/or plasma biomarkers (e.g., MCP-1, FGF23, IGF-1, TGFB1, homocysteine, glutathione, or combinations thereof) can be used to assess a treatment response (e.g., biomarker changes).

In some cases, methods described herein can include monitoring a mammal being treated as described herein for toxicity. The level of toxicity, if any, can be determined by assessing a mammal's clinical signs and symptoms before and after administering a known amount of a particular composition. It is noted that the effective amount of a particular composition administered to a mammal can be adjusted according to a desired outcome as well as the mammal's response and level of toxicity.

The invention will be further described in the following examples, which do not limit the scope of the invention described in the claims.

### EXAMPLES

### Example 1: Role of Betaine in ADPKD

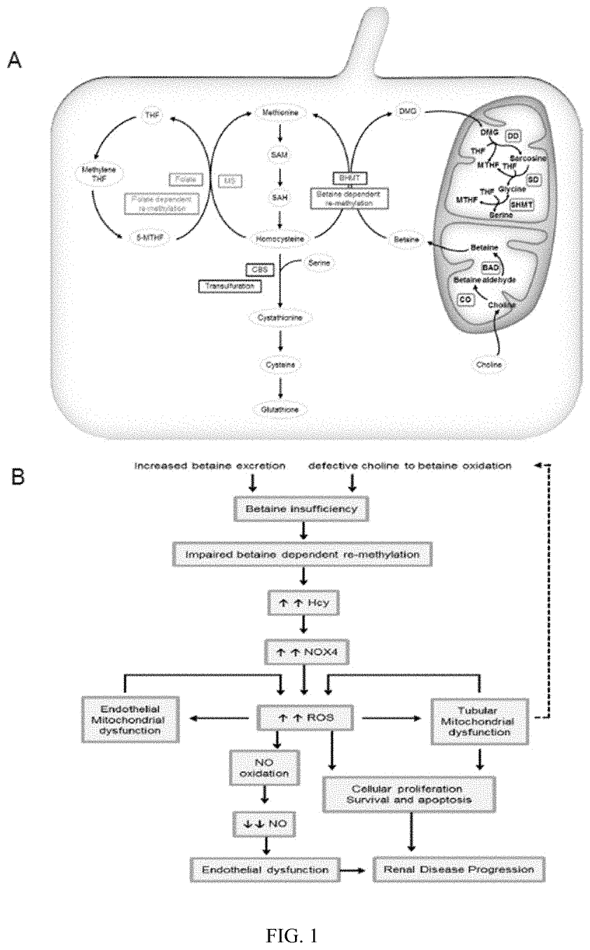

Homocysteine (Hcy) is a thiol-containing amino acid biosynthesized from methionine. Once generated, Hcy can be metabolized to cysteine by the enzyme cystathione β-synthase (CBS) through the transsulfuration pathway, or re-methylated to methionine via the folate-dependent pathway by methionine synthase (MS) or via the betaine dependent pathway by betaine-homocysteine methyltransferase (BHMT) (FIG. 1A).

To explore the mechanisms underlying the increase in Hcy in ADPKD, a targeted analysis of the Hcy pathway was performed. It was found that urine Hcy and plasma methionine and cystathionine levels were similar between patients with ADPKD and controls. Furthermore, folate and vitamin B12 levels were similar between patients with ADPKD and controls, arguing against a defect in the transulfuration or folate-dependent re-methylation pathways. In addition to folate dependent re-methylation, there is an alternate pathway which is carried out by BHMT using betaine (trimethylglycine) as methyl donor, producing methionine and DMG (FIG. 1A). Betaine is freely filtered, but renal excretion is low, as more than 98% is reabsorbed in the renal tubules. Interestingly, it was found that young patients with ADPKD had higher urine excretion of betaine compared to controls. Despite this, plasma betaine levels were similar between the groups suggesting that patients with ADPKD may have increased urine betaine loss rather than increased betaine production. Kidney tissue from Pkd1^{RC/RC} animals was investigated and it was found that betaine concentration decreased and inversely correlated with urine betaine, further supporting an increase in betaine loss and/or defective conversion of choline to betaine. The increase in betaine loss observed in ADPKD may result from a decreased medullary osmolality and renal concentrating defects or impaired betaine re-uptake. The increase in urine betaine loss may disrupt betaine-dependent re-methylation by decreasing betaine availability and leading to increased Hcy levels. It was found that urine excretion of betaine was correlated with plasma Hcy, suggesting that in ADPKD, mild hyperhomocysteinemia may be related to increased betaine loss. Likewise, a disruption of mitochondrial choline oxidation to betaine may lead to tissue betaine insufficiency and increased Hcy. Increased urine betaine excretion was also associated with increased urine DMG excretion in patients with ADPKD. Additionally, tissue and urine DMG concentration trended to be lower in Pkd1^{RC/RC} animals compared to controls, which may suggest the concentrations of these metabolites may change with different stages of the disease. Thus, early in ADPKD, increased plasma Hcy levels may be due, at least in part, to increased urinary betaine excretion and plasma DMG accumulation leading to decreased betaine-dependent re-methylation.

These results suggest that betaine supplementation can be used to treat PKD.

### Example 2: Betaine Supplementation Ameliorates Renal Disease Severity in Experimental ADPKD

This Example evaluates whether betaine supplementation can increase renal betaine concentration and ameliorate disease progression in murine ADPKD.

### Methods

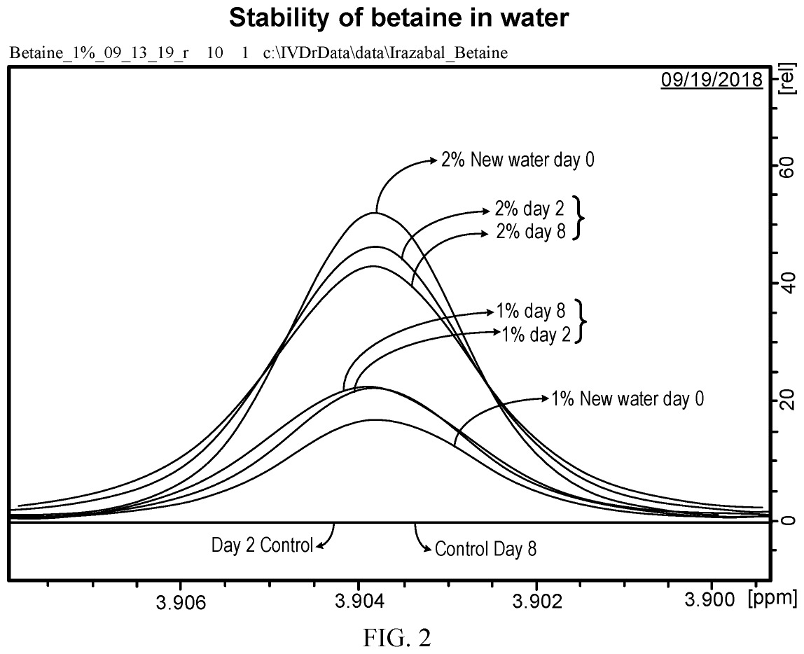

One month old Pkd1^{RC/RC} mice were divided into three groups and started treatment with regular water or regular water supplemented with 1% or 2% betaine (FIG. 2) for 5 months (n=16 per group). All mice were euthanized at 6 months of age, and kidneys harvested. Cystic index was determined from histological sections. 1H-NMR-based metabolomics analysis was performed from tissue urine and plasma samples.

Intrarenal microvasculature was quantified using a capillary index. Capillaries in the cortex were identified by the presence of lumen, red blood cells, and/or an endothelial cell lining. For the PKD animals, the number of capillaries is calculated adjusting for the cystic area.

Blood pressure was determined during the same time frame (9-11 am) in all animals in all groups, using the CODA non-invasive blood pressure system (Kent Scientific). Briefly, mice were guided into a plastic mouse holder and were left to acclimate to the holder for 5 minutes. Ten blood pressure (BP) measurements within 10 minutes were obtained by tail cuff.

### Results

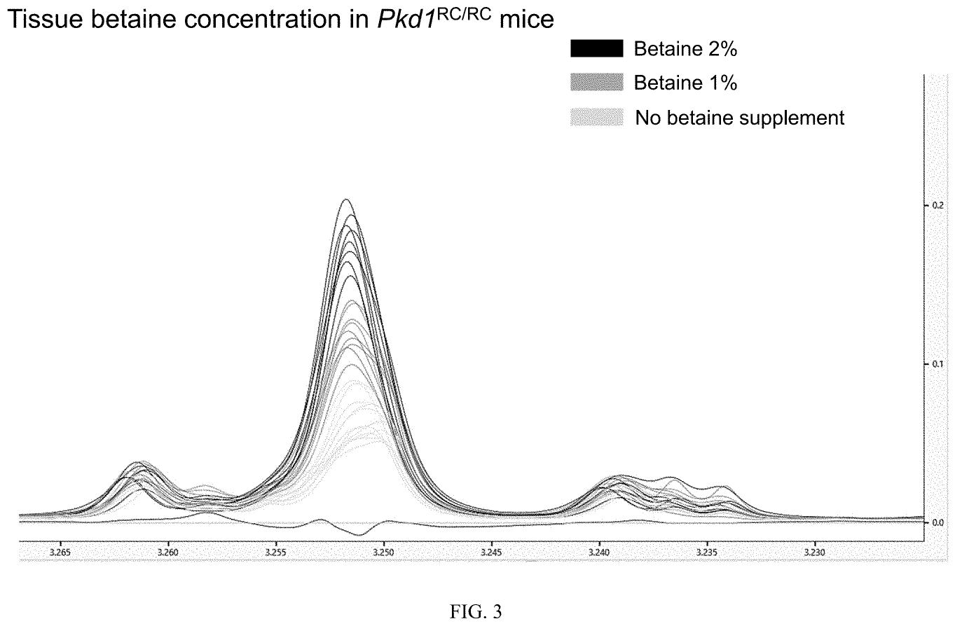

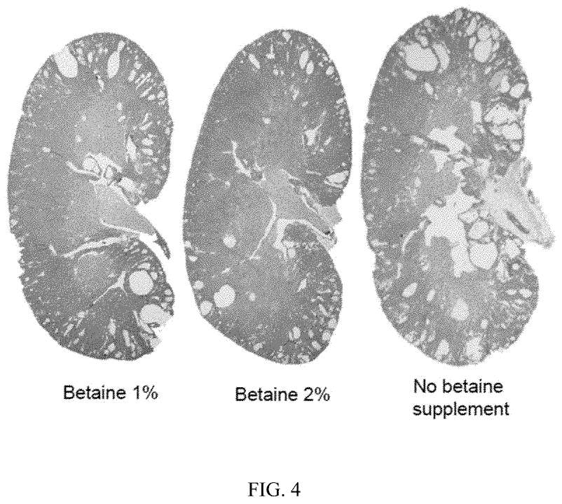

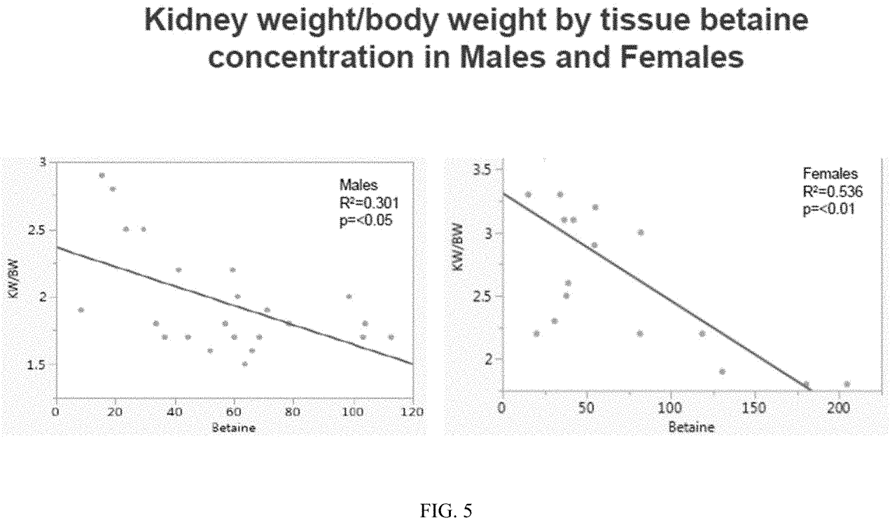

One and 2% betaine supplementation increased plasma and tissue betaine concentrations (p<0.001) (FIG. 3), and reduced kidney/body weight to 1.82 and 1.85 vs 2.25 (p<0.01), and cystic index to 11.1 and 9.4 vs 21.1 (p<0.01) (FIG. 4). Tissue betaine concentrations correlated inversely with kidney/body weight (R²=0.386, p<0.01) (FIG. 5).

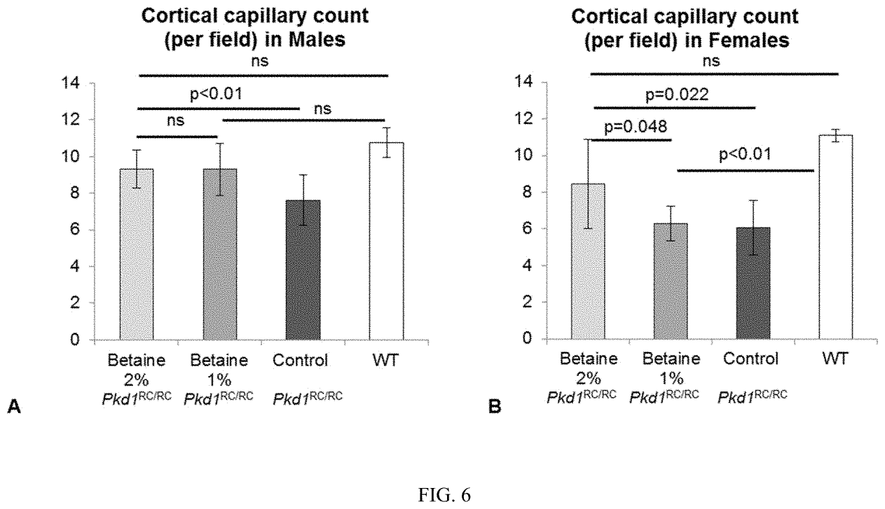

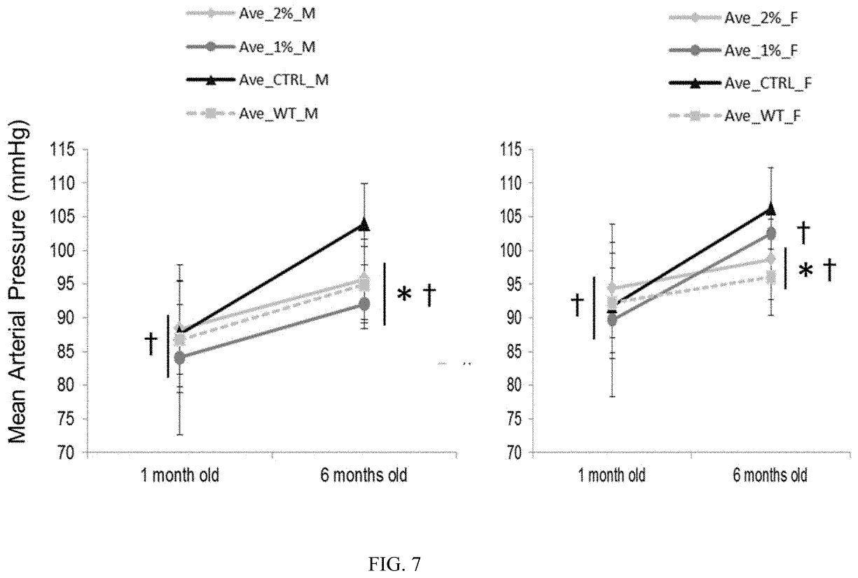

Metabolomics analyses from tissue and plasma identified significant differences in mitochondrial fatty acid oxidation and TCA cycle pathways among the groups. One and 2% betaine supplementation preserved the capillary count in Pkd1^{RC/RC} male animals, but in female animals, although 2% betaine supplementation preserved capillary count, 1% betaine supplementation was not effective in preserving the intrarenal capillaries (FIG. 6). One and 2% betaine supplementation preserved MAP within WT levels in Pkd1^{RC/RC} animals (FIG. 7).

These results suggest that betaine supplementation can be used to treat PKD. For example, administering betaine to a mammal having PKD can reduce body weight and/or can decrease a cystic index within the mammal. For example, administering betaine to a mammal having PKD can preserve the vasculature (e.g., capillary count) and/or can reduce blood pressure within the mammal.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a mammal having a polycystic kidney disease (PKD), wherein said method comprises administering a composition comprising one or more betaines to said mammal to reduce a cystic index in said mammal.

2. The method of claim 1, wherein said method comprises identifying said mammal as being in need of a treatment for said PKD.

3. The method of claim 1, wherein said mammal is a human.

4. The method of claim 1, wherein PKD is an autosomal dominant PKD (ADPKD).

5. The method of claim 1, wherein said composition comprises trimethylglycine.

6. The method of claim 5, wherein said composition comprises from about 1 gram to about 20 g of said trimethylglycine.

7. The method of claim 1, wherein said composition comprises one or more vasopressin receptor antagonists.

8. The method of claim 7, wherein said composition comprises tolvaptan or lixivaptan.

9. A method for treating a mammal having a PKD, wherein said method comprises administering a composition comprising one or more betaines to said mammal to reduce a symptom of said PKD in said mammal.

10. The method of claim 9, wherein said method comprises identifying said mammal as being in need of a treatment for said PKD.

11. The method of claim 9, wherein said mammal is a human.

12. The method of claim 9, wherein PKD is an ADPKD.

13. The method of claim 9, wherein said symptom is selected from the group consisting of back pain, side pain, headache, a feeling of fullness, an enlarged kidney, blood in the urine, high blood pressure, kidney failure, mitral valve prolapse, diverticulosis, brain aneurysm, and endothelial dysfunction (ED).

14. The method of claim 9, wherein said composition comprises trimethylglycine.

15. The method of claim 14, wherein said composition comprises from about 1 g to about 20 g of said trimethylglycine.

16. The method of claim 9, wherein said composition comprises one or more vasopressin receptor antagonists.

17. The method of claim 16, wherein said composition comprises tolvaptan or lixivaptan.

18. A method for treating a mammal having a PKD, wherein said method comprises administering a composition comprising one or more betaines to said mammal to increase a concentration of renal betaine in said mammal.

19. The method of claim 18, wherein said method comprises identifying said mammal as being in need of a treatment for said PKD.

20. The method of claim 18, wherein said mammal is a human.
